(19) United States
(12) Patent Application Publication
(10) Pub. No.: US 2024/0009384 A1
(43) Pub. Date: Jan. 11, 2024

(54) System and Method for Detecting Priming of a Fluid Path of a Drug Delivery Device

(71) Applicant: Becton, Dickinson and Company, Franklin Lakes, NJ (US)

(72) Inventors: Danielle Aboud, Dublin (IE); Steve Beguin, Rathdrum (IE)

(21) Appl. No.: 17/860,525

(22) Filed: Jul. 8, 2022

(51) Int. Cl. A61M 5/142; A61M 5/158

(52) U.S. Cl. CPC A61M 5/14248; A61M 5/158; A61M 5/14244; A61M 2005/14252; A61M 2005/1585

(57) ABSTRACT

A drug delivery device includes a housing, a reservoir positioned within the housing and configured to receive a fluid, a fluid line in fluid communication with the reservoir, a delivery sub-system configured to deliver a fluid from the reservoir to the fluid line, an insertion mechanism including a cannula in fluid communication with the fluid line, with the insertion mechanism configured to move the cannula from a retracted position where the cannula is positioned within the housing to an extended position where at least a portion of the cannula is positioned outside of the housing, a sensor configured to detect when air is present within the fluid line, and control electronics configured to actuate the insertion mechanism based on a signal from the sensor.

### BACKGROUND OF THE INVENTION

### Field of the Invention

The present disclosure relates to a system and method for detecting priming of a fluid path of a drug delivery device.

### Description of Related Art

Wearable medical devices, such as automatic injectors or infusion devices, have the benefit of providing therapy to the patient at a location remote from a clinical facility and/or while being worn discretely under the patient's clothing. The wearable medical device can be applied to the patient's skin and configured to automatically deliver a dose of a pharmaceutical composition within a predetermined time period after applying the wearable medical device to the patient's skin. After the device delivers the pharmaceutical composition to the patient, the patient may subsequently remove and dispose of the device.

In order to achieve repeatable and accurate performance of the wearable medical device, such as accurate dosing, the device must be primed to remove air from the fluid path. The device may be primed manually or as part of a sequence of device activation steps performed by a patient or healthcare technician.

### SUMMARY OF THE INVENTION

In one aspect or embodiment, a drug delivery device includes a housing, a reservoir positioned within the housing and configured to receive a fluid, a fluid line in fluid communication with the reservoir, a delivery sub-system configured to deliver a fluid from the reservoir to the fluid line, an insertion mechanism including a cannula in fluid communication with the fluid line, with the insertion mechanism configured to move the cannula from a retracted position where the cannula is positioned within the housing to an extended position where at least a portion of the cannula is positioned outside of the housing, a sensor configured to detect when air is present within the fluid line, and control electronics configured to actuate the insertion mechanism based on a signal from the sensor.

The drug delivery device may include a hydrophobic membrane in fluid communication with the fluid line, with the hydrophobic membrane configured to allow air to be expelled from the fluid line. The sensor may be positioned upstream of the hydrophobic membrane. The sensor may be positioned downstream of the hydrophobic membrane. The control electronics may include a microcontroller. The cannula may include a needle and a catheter, with the insertion mechanism configured to place the catheter into a patient via the needle. The sensor may include a pressure sensor. The sensor may include a fluid detection sensor. The sensor may include an electronic switch having an open position when in fluid communication with air and a closed position when in fluid communication with liquid. The insertion mechanism may include a solenoid actuator. The insertion mechanism may include a piezoelectric actuator. The drug delivery device may further include a power source. The control electronics may be configured to determine a presence of air by evaluating a pressure drop when fluid is pumped in the fluid line between the delivery sub-system and the cannula and comparing the pressure drop with a threshold value, with the pressure drop determined based on data from the sensor. The control electronics may be configured to determine a presence of air by evaluating a pressure drop when fluid is delivered to the cannula and comparing the pressure drop with a threshold value, with the pressure drop determined based on data from the sensor.

In a further aspect or embodiment, a method of utilizing the drug delivery device of any of the aspects or embodiments discussed above includes: actuating the drug delivery device; delivering fluid from the reservoir to the fluid line; determining whether air is present within the fluid line; sending a signal to the control electronics when air is removed from the fluid line to a predetermined acceptable limit; and automatically actuating the insertion mechanism to move the cannula from the retracted position to the extended position.

In a further aspect or embodiment, a drug delivery device includes a housing, a fluid line, a delivery sub-system configured to deliver a fluid via the fluid line into a patient, and a sensor configured to detect when air is present within the fluid line.

The drug delivery device may further include a reservoir positioned within the housing and configured to receive a fluid, with the fluid line in fluid communication with the reservoir, and with the delivery sub-system configured to deliver a fluid from the reservoir to the fluid line. The drug delivery device may further include an insertion mechanism including a cannula in fluid communication with the fluid line, with the insertion mechanism configured to move the cannula from a retracted position where the cannula is positioned within the housing to an extended position where at least a portion of the cannula is positioned outside of the housing. The drug delivery device may further include control electronics configured to actuate an insertion mechanism based on a signal from the sensor. The sensor may be near the distal end of the fluid line. The sensor may be near the proximal end of the fluid line.

The device may include one or several of the following features, taken individually or accordingly to all technical possible combinations:
a drug delivery device may include a housing; a reservoir positioned within the housing and configured to receive a fluid; a fluid line in fluid communication with the reservoir; a delivery sub-system configured to deliver a fluid from the reservoir to the fluid line; an insertion mechanism comprising a cannula in fluid communication with the fluid line, the insertion mechanism configured to move the cannula from a retracted position where the cannula is positioned within the housing to an extended position where at least a portion of the cannula is positioned outside of the housing; a sensor configured to detect when air is present within the fluid line; and control electronics configured to actuate the insertion mechanism based on a signal from the sensor;
the device may further include a hydrophobic membrane in fluid communication with the fluid line, the hydrophobic membrane configured to allow air to be expelled from the fluid line;
the sensor may be positioned upstream of the hydrophobic membrane;
the sensor may be positioned downstream of the hydrophobic membrane;
the control electronics may include a microcontroller;
the cannula may include a needle and a catheter, the insertion mechanism configured to place the catheter into a patient via the needle;
the sensor may include a pressure sensor;
the control electronics may be configured to determine a presence of air by evaluating a pressure drop when fluid is pumped in the fluid line between the delivery sub-system and the cannula and comparing the pressure drop with a threshold value, and where the pressure drop is determined based on data from the sensor;
control electronics may be configured to determine a presence of air by evaluating a pressure drop when fluid is delivered to the cannula and comparing the pressure drop with a threshold value, and where the pressure drop is determined based on data from the sensor;
the sensor may include a fluid detection sensor;
the sensor may include an electronic switch having an open position when in fluid communication with air and a closed position when in fluid communication with liquid;
the insertion mechanism may include a solenoid actuator;
the insertion mechanism may include a piezoelectric actuator; and
the device may further include a power source.

### BRIEF DESCRIPTION OF THE DRAWINGS

The above-mentioned and other features and advantages of this disclosure, and the manner of attaining them, will become more apparent and the disclosure itself will be better understood by reference to the following descriptions of embodiments of the disclosure taken in conjunction with the accompanying drawings.

FIG. 1 is a perspective view of a drug delivery device according to one aspect or embodiment of the present application.

FIG. 2 is a perspective view of the drug delivery device of FIG. 1, with a top cover removed.

FIG. 3 is a schematic view of the drug delivery device of FIG. 1.

FIG. 4 is a schematic view of a system for priming a fluid path of a drug delivery device according to one aspect or embodiment of the present application.

FIG. 5 is a schematic view of a system for priming a fluid path of a drug delivery device according to a further aspect or embodiment of the present application.

Corresponding reference characters indicate corresponding parts throughout the several views. The exemplifications set out herein illustrate exemplary embodiments of the disclosure, and such exemplifications are not to be construed as limiting the scope of the disclosure in any manner.

### DETAILED DESCRIPTION OF THE INVENTION

Spatial or directional terms, such as “left”, “right”, “inner”, “outer”, “above”, “below”, and the like, are not to be considered as limiting as the invention can assume various alternative orientations.

All numbers used in the specification and claims are to be understood as being modified in all instances by the term “about”. By “about” is meant a range of plus or minus ten percent of the stated value. As used in the specification and the claims, the singular form of “a”, “an”, and “the” include plural referents unless the context clearly dictates otherwise. The terms “first”, “second”, and the like are not intended to refer to any particular order or chronology, but instead refer to different conditions, properties, or elements. By “at least” is meant “greater than or equal to”.

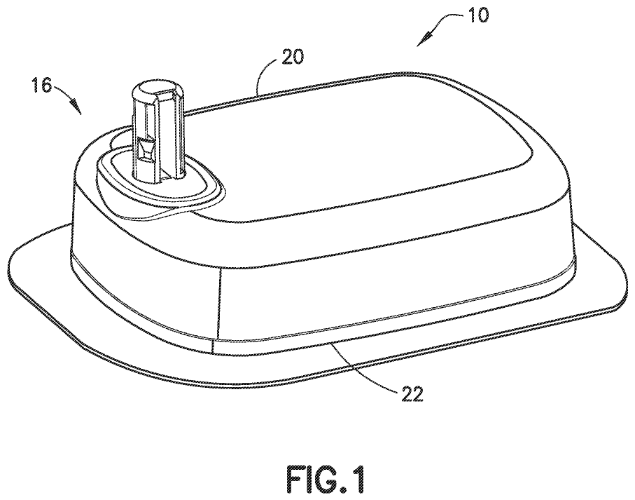

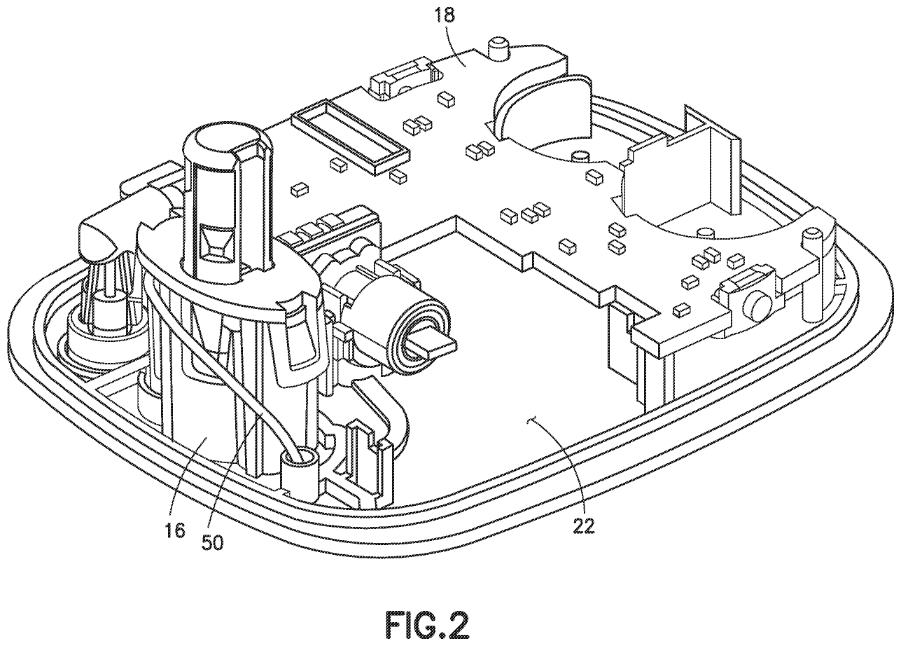

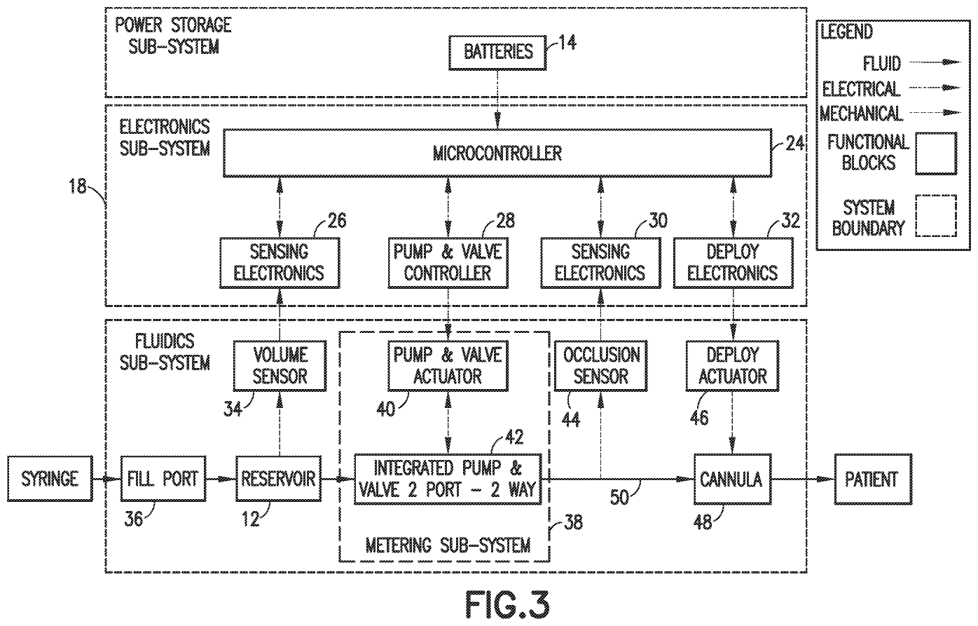

Referring to FIGS. 1-3, a drug delivery device 10 includes a reservoir 12, a power source 14, an insertion mechanism 16, control electronics 18, a cover 20, and a base 22. In one aspect or embodiment, the drug delivery device 10 is a wearable automatic injector, such as an insulin or bone marrow stimulant delivery device. The drug delivery device 10 may be mounted onto the skin of a patient and triggered to inject a pharmaceutical composition from the reservoir 12 into the patient. The drug delivery device 10 may be pre-filled with the pharmaceutical composition, or it may be filled with the pharmaceutical composition by the patient or medical professional prior to use.

The drug delivery device 10 is configured to deliver a dose of a pharmaceutical composition, e.g., any desired medicament, into the patient's body by a subcutaneous injection at a controlled injection rate. Exemplary time durations for the delivery achieved by the drug delivery device 10 may range from about 5 minutes to about 60 minutes, but are not limited to this exemplary range. Exemplary volumes of the pharmaceutical composition delivered by the drug delivery device 10 may range from about 0.1 milliliters to about 10 milliliters, but are not limited to this exemplary range. The volume of the pharmaceutical composition delivered to the patient may be adjusted.

Referring again to FIGS. 1-3, in one aspect or embodiment, the power source 14 is a DC power source including one or more batteries. The control electronics 18 include a microcontroller 24, sensing electronics 26, a pump and valve controller 28, sensing electronics 30, and deployment electronics 32, which control the actuation of the drug delivery device 10. The drug delivery device 10 includes a fluidics sub-system that includes the reservoir 12, a volume sensor 34 for the reservoir 12, a reservoir fill port 36, and a delivery or metering sub-system 38 including a pump and valve actuator 40 and a pump and valve mechanism 42. The fluidic sub-system may further include an occlusion sensor 44, a deploy actuator 46, a cannula 48 for insertion into a patient's skin, and a fluid line 50 in fluid communication with the reservoir 12 and the cannula 48. In one aspect or embodiment, the insertion mechanism 16 is configured to move the cannula 48 from a retracted position positioned entirely within the device 10 to an extended position where the cannula 48 extends outside of the device 10. The drug delivery device 10 may operate in the same manner as discussed in U.S. Pat. No. 10,449,292 to Pizzochero et al., incorporated herein by reference.

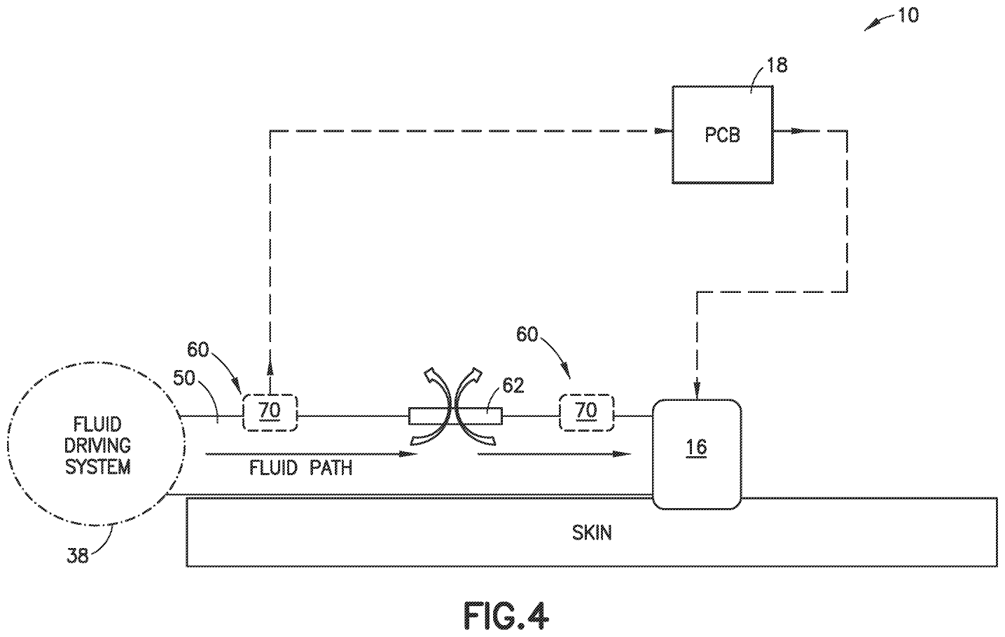

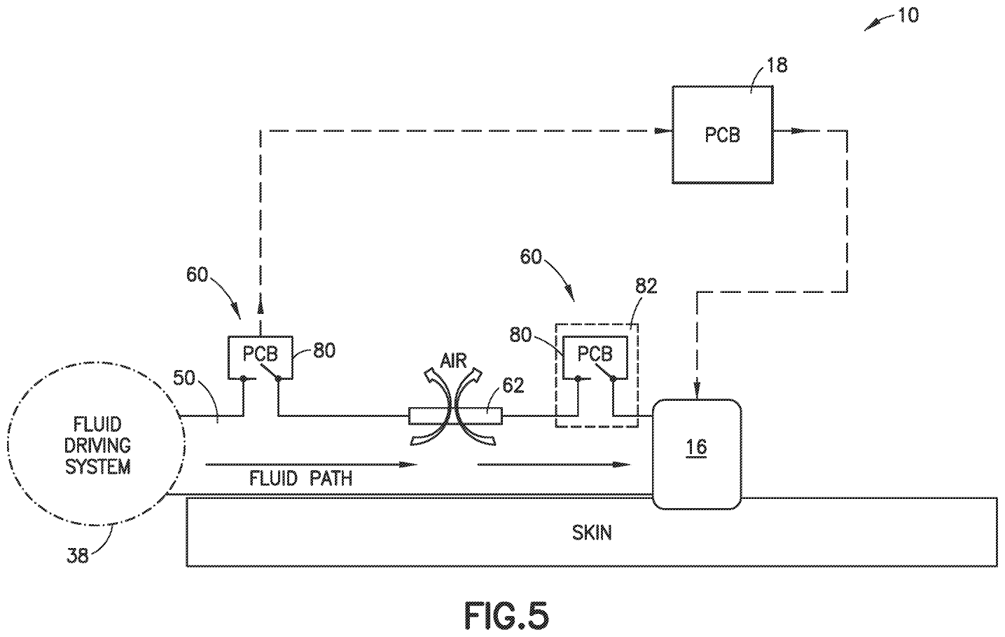

Referring to FIGS. 4 and 5, in one aspect or embodiment of the present application, the drug delivery device 10 includes a sensor 60 configured to detect when air is present within the fluid line 50, with the control electronics 18 configured to actuate the insertion mechanism 16 based on a signal from the sensor 60. In some aspects or embodiments, the sensor 60 enables automatic priming of the device 10 followed by automatic insertion of the cannula 48 into subcutaneous tissue and requiring only one interaction step from a patient or healthcare technician.

Referring again to FIGS. 4 and 5, in one aspect or embodiment, the drug delivery device 10 includes a hydrophobic membrane 62 in fluid communication with the fluid line 50, with the hydrophobic membrane 62 configured to allow air to be expelled from the fluid line 50. As shown in FIGS. 4 and 5, the sensor 60 may be positioned upstream of the hydrophobic membrane 62 or may be positioned downstream of the hydrophobic membrane 62. The sensor 60 may be positioned closer to the delivery sub-system 38 than the insertion mechanism 16 or may be positioned closer to the insertion mechanism 16 than the delivery sub-system 38. The sensor may be positioned closer to a distal end of the fluid line 50 than a proximal end of the fluid line or may be positioned closer to the proximal end of the fluid line 50 than the distal end of the fluid line 50. As noted above, the control electronics 18 may include the microcontroller 24, although other processors and arrangements may be utilized. The cannula 48 may include a needle and/or a catheter (not shown), with the insertion mechanism 16 configured to place the catheter into a patient via the needle and subsequently retract the needle leaving the catheter within the patient. The catheter may be a soft catheter.

Referring to FIG. 4, in one aspect or embodiment, the sensor 60 includes a pressure sensor 70. Air is less viscous than water and other liquids and, due to this property difference, air within the fluid line may be detected via the pressure sensor 70 when the fluid line 50 has been expelled of all or mostly all of any air within the fluid line 50. When air is no longer detected or at a predetermined acceptable limit, the pressure sensor 70 is configured to send a signal to the control electronics 18 and/or the insertion mechanism 16 to activate the insertion mechanism. The sensor 60 may also be or further include a fluid detection sensor, such an optical or capacitive fluid sensor. In one aspect or embodiment, the control electronics 18 is configured to determine a presence of air by evaluating a pressure drop when fluid is pumped in the fluid line 50 between the delivery sub-system 38 and the cannula 48 and comparing the pressure drop with a threshold value, with the pressure drop determined based on data from the sensor 60. In one aspect or embodiment, the control electronics 18 is configured to determine a presence of air by evaluating a pressure drop when fluid is delivered to the cannula 48 and comparing the pressure drop with a threshold value, with the pressure drop determined based on data from the sensor 60. In some aspects or embodiments, a pressure drop when the fluid line 50 has an unacceptable level of air within the fluid line 50 is determined experimentally and/or theoretically, which can be correlated to a range or predetermined acceptable limit for air within the fluid line 50. Based on data from the sensor 60, the control electronics 18 can compare pressure values and/or drops within the fluid line 50 with known values that indicate the presence or absence of air within the fluid line 50.

Referring to FIG. 5, in one aspect or embodiment, the sensor 60 includes an electronic switch 80 having an open position when in fluid communication with air and a closed position when in fluid communication with liquid. The electronic switch 80 is configured to be closed by conductive fluid, such as liquid medicament within the reservoir 12 and delivered to the fluid line 50, coming into contact with the electronic switch 80. The electronic switch 80 may be positioned in a channel 82 positioned off of the fluid line 50, although other suitable arrangements may be utilized.

In some aspects or embodiments, the insertion mechanism 16 includes a solenoid actuator or a piezoelectric actuator (not shown). Further, in some aspects or embodiments, an electronic signal from the sensor 60 is sent via power cables through tubing of an infusion-type device or through flex PCB.

In one aspect or embodiment, a method of utilizing the drug delivery device 10 includes: actuating the drug delivery device 10; delivering fluid from the reservoir 12 to the fluid line 50; determining whether air is present within the fluid line 50; sending a signal to the control electronics 18 when air is removed from the fluid line 50; and automatically actuating the insertion mechanism 16 to move the cannula 48 from the retracted position to the extended position.

Although the invention has been described in detail for the purpose of illustration based on what is currently considered to be the most practical and preferred embodiments, it is to be understood that such detail is solely for that purpose and that the invention is not limited to the disclosed embodiments, but, on the contrary, is intended to cover modifications and equivalent arrangements that are within the spirit and scope of the appended claims. For example, it is to be understood that the present invention contemplates that, to the extent possible, one or more features of any embodiment can be combined with one or more features of any other embodiment.

The invention claimed is:

1. A drug delivery device comprising:
a housing;
a reservoir positioned within the housing and configured to receive a fluid;
a fluid line in fluid communication with the reservoir;
a delivery sub-system configured to deliver a fluid from the reservoir to the fluid line;
an insertion mechanism comprising a cannula in fluid communication with the fluid line, the insertion mechanism configured to move the cannula from a retracted position where the cannula is positioned within the housing to an extended position where at least a portion of the cannula is positioned outside of the housing;
a sensor configured to detect when air is present within the fluid line; and
control electronics configured to actuate the insertion mechanism based on a signal from the sensor.

2. The drug delivery device of claim 1, further comprising a hydrophobic membrane in fluid communication with the fluid line, the hydrophobic membrane configured to allow air to be expelled from the fluid line.

3. The drug delivery device of claim 2, wherein the sensor is positioned upstream of the hydrophobic membrane.

4. The drug delivery device of claim 2, wherein the sensor is positioned downstream of the hydrophobic membrane.

5. The drug delivery device of claim 1, wherein the control electronics comprises a microcontroller.

6. The drug delivery device of claim 1, wherein the cannula comprises a needle and a catheter, the insertion mechanism configured to place the catheter into a patient via the needle.

7. The drug delivery device of claim 1, wherein the sensor comprises a pressure sensor.

8. The drug delivery device of claim 1, wherein the control electronics is configured to determine a presence of air by evaluating a pressure drop when fluid is pumped in the fluid line between the delivery sub-system and the cannula and comparing the pressure drop with a threshold value, and wherein the pressure drop is determined based on data from the sensor.

9. The drug delivery device of claim 1, wherein the control electronics is configured to determine a presence of air by evaluating a pressure drop when fluid is delivered to the cannula and comparing the pressure drop with a threshold value, and wherein the pressure drop is determined based on data from the sensor.

10. The drug delivery device of claim 1, wherein the sensor comprises a fluid detection sensor.

11. The drug delivery device of claim 1, wherein the sensor comprises an electronic switch having an open position when in fluid communication with air and a closed position when in fluid communication with liquid.

12. The drug delivery device of claim 1, wherein the insertion mechanism comprises a solenoid actuator.

13. The drug delivery device of claim 1, wherein the insertion mechanism comprises a piezoelectric actuator.

14. The drug delivery device of claim 1, further comprising a power source.

15. A method of utilizing the drug delivery device of claim 1, the method comprising:
actuating the drug delivery device;
delivering fluid from the reservoir to the fluid line;
determining whether air is present within the fluid line;
sending a signal to the control electronics when air is removed from the fluid line to a predetermined acceptable limit; and
automatically actuating the insertion mechanism to move the cannula from the retracted position to the extended position.

16. A drug delivery device comprising:
a housing;
a fluid line;
a delivery sub-system configured to deliver a fluid via the fluid line into a patient; and
a sensor configured to detect when air is present within the fluid line.

17. The drug delivery device of claim 16, further comprising a reservoir positioned within the housing and configured to receive a fluid, wherein the fluid line is in fluid communication with the reservoir, and wherein the delivery sub-system is configured to deliver a fluid from the reservoir to the fluid line.

18. The drug delivery device of claim 16, further comprising:
an insertion mechanism comprising a cannula in fluid communication with the fluid line, the insertion mechanism configured to move the cannula from a retracted position where the cannula is positioned within the housing to an extended position where at least a portion of the cannula is positioned outside of the housing; and
control electronics configured to actuate an insertion mechanism based on a signal from the sensor.

19. The drug delivery device of claim 16, wherein the sensor is near the distal end of the fluid line.

20. The drug delivery device of claim 16, wherein the sensor is near the proximal end of the fluid line.
